(19) United States
(12) Patent Application Publication
(10) Pub. No.: US 2026/0141996 A1
(43) Pub. Date: May 21, 2026

(54) INFORMATION PROCESSING APPARATUS, INFORMATION PROCESSING METHOD, AND NON-TRANSITORY RECORDING MEDIUM

(71) Applicant: NEC Corporation, Tokyo (JP)

(72) Inventors: Daisaku SHIBATA, Tokyo (JP); Masanori TSUJIKAWA, Tokyo (JP)

(73) Assignee: NEC Corporation, Tokyo (JP)

(21) Appl. No.: 19/394,382

(22) Filed: Nov. 19, 2025

(30) Foreign Application Priority Data
Nov. 20, 2024 (JP) 2024-202638

(51) Int. Cl. G16H 10/20; G06N 20/00; G16H 10/60

(52) U.S. Cl. CPC G16H 10/20; G06N 20/00; G16H 10/60

(57) ABSTRACT

An information processing apparatus includes an acquisition unit for acquiring medical care information on one or more subjects, a structuring unit for generating structured medical care information by structuring at least a part of the medical care information, a first generation unit for generating input information including the structured medical care information and a query, and a second generation unit for generating an answer to the query related to the one or more subjects with reference to an output from a generative model having received the input information.

### INCORPORATION BY REFERENCE

This application is based upon and claims the benefit of priority from Japanese patent application No. 2024-202638, filed on Nov. 20, 2024, the disclosure of which is incorporated herein in its entirety by reference.

### TECHNICAL FIELD

The present disclosure relates to an information processing apparatus, an information processing method, and a non-transitory recording medium.

### BACKGROUND ART

There is known a technique for performing estimation related to a patient using a machine learning technique. For example, a technique for performing matching between a clinical trial and a patient using a large language models (LLMs) is disclosed in “Qiao Jin et. al. Matching Patients to Clinical Trials with Large Language Models, arXiv:2307.15051”.

### SUMMARY

In general, as an attendance period or a hospital stay period of a patient becomes longer, medical care information on the patient also increases in amount. The technique described in “Qiao Jin et. al. Matching Patients to Clinical Trials with Large Language Models, arXiv:2307.15051” causes a problem that estimation accuracy is likely to decrease because medical care information on a patient is directly input to a large language model.

The present disclosure has been made in view of the above problems, and an example object thereof is to provide a technique capable of performing estimation related to a patient with high accuracy while using a generative model.

An information processing apparatus according to an example aspect of the present disclosure includes acquisition means for acquiring medical care information on one or more subjects, structuring means for generating structured medical care information by structuring at least a part of the medical care information, first generation means for generating input information including the structured medical care information and a query, and second generation means for generating an answer to the query related to the one or more subjects with reference to output from a generative model having received the input information.

An information processing method according to an example aspect of the present disclosure uses one or more processors to perform processing including acquiring medical care information on one or more subjects, generating structured medical care information by structuring at least a part of the medical care information, generating input information including the structured medical care information and a query, and generating an answer to the query regarding the one or more subjects with reference to output from a generative model having received the input information.

A program according to an example aspect of the present disclosure is a program for causing a computer to function as an information processing apparatus, the program causing the computer to function as acquisition means for acquiring medical care information on one or more subjects, structuring means for generating structured medical care information by structuring at least a part of the medical care information, first generation means for generating input information including the structured medical care information and a query, and second generation means for generating an answer to the query related to the one or more subjects with reference to output from a generative model having received the input information.

The example aspects of the present disclosure achieve exemplary effect capable of estimation related to a patient with high accuracy while a generative model is used.

### BRIEF DESCRIPTION OF DRAWINGS

FIG. 1 is a block diagram illustrating a configuration of an information processing apparatus according to the present disclosure;

FIG. 2 is a flowchart illustrating a flow of an information processing method according to the present disclosure;

FIG. 3 is a block diagram illustrating a configuration of an information processing system according to the present disclosure;

FIG. 4 is a diagram for illustrating information referred to by the information processing system according to the present disclosure;

FIG. 5 is a flowchart illustrating a processing flow example in the information processing system according to the present disclosure;

FIG. 6 is a diagram for illustrating an example of data processing in the information processing system according to the present disclosure;

FIG. 7 is a diagram for illustrating an example of data processing in the information processing system according to the present disclosure;

FIG. 8 is a diagram for illustrating an example of data processing in the information processing system according to the present disclosure;

FIG. 9 is a diagram for illustrating an example of data processing in the information processing system according to the present disclosure;

FIG. 10 is a diagram for illustrating an example of data processing in the information processing system according to the present disclosure;

FIG. 11 is a diagram illustrating a display example using the information processing system according to the present disclosure;

FIG. 12 is a flowchart illustrating a processing flow example in the information processing system according to the present disclosure;

FIG. 13 is a block diagram illustrating a configuration of the information processing system according to the present disclosure; and

FIG. 14 is a block diagram illustrating a configuration of a computer that functions as the information processing apparatus according to the present disclosure.

### EXAMPLE EMBODIMENT

Hereinafter, example embodiments of the present invention will be exemplified. However, the present invention is not limited to the following exemplary example embodiments, and various modifications can be made within a scope described in the claims. For example, example embodiments obtained by appropriately combining technologies (some or all of things or methods) adopted in the following exemplary example embodiments can also be included in the scope of the present invention. Example embodiments obtained by appropriately omitting some of the technologies adopted in the following exemplary example embodiments can also be included in the scope of the present invention. Effects mentioned in the following exemplary example embodiments are examples of effects expected in the exemplary example embodiments, and do not define extension of the present invention. In other words, example embodiments that do not provide the effects mentioned in the following exemplary example embodiments can also be included in the scope of the present invention.

### First Example Embodiment

A first exemplary example embodiment that is an example of an example embodiment of the present invention will be described in detail with reference to the drawings. The present exemplary example embodiment is a basic form of each exemplary example embodiment to be described below. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technology illustrated in the drawings referred to for describing the present exemplary example embodiment can also be adopted in another exemplary example embodiment included in the present disclosure within a range in which no particular technical problem occurs.

### Configuration of Information Processing Apparatus 1

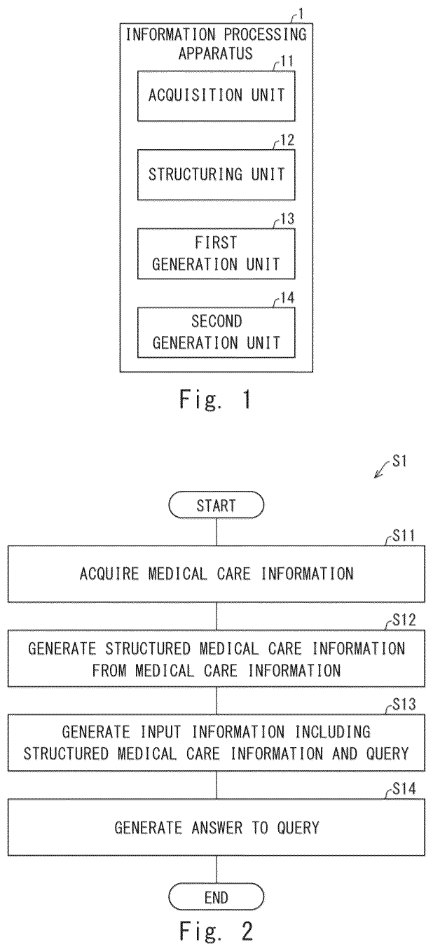

A configuration of an information processing apparatus 1 according to the present exemplary example embodiment will be described with reference to FIG. 1. FIG. 1 is a block diagram illustrating a configuration of the information processing apparatus 1. As illustrated in FIG. 1, the information processing apparatus 1 includes an acquisition unit 11, a structuring unit 12, a first generation unit 13, and a second generation unit 14.

### Acquisition Unit 11

The acquisition unit 11 acquires medical care information on one or more subjects. Here, the medical care information may include various types of information extracted from a medical record (including an electronic medical record) of a subject (target patient). For example, the medical care information may include information on the subject, such as an initial medical interview, a progress record, an image interpretation report, and a nursing record. However, these examples do not limit the present exemplary example embodiment.

### Structuring Unit 12

The structuring unit 12 generates structured medical care information by structuring at least a part of the medical care information acquired by the acquisition unit 11. Specific structuring processing performed by the structuring unit 12 does not limit the present exemplary example embodiment, and processing below may be performed, for example.
Example 1: A plurality of entities is extracted from at least a part of the medical care information, and one or more triplets including the plurality of entities and a relation between the plurality of entities are generated as the structured medical care information.
Example 2: A plurality of entities is extracted from at least a part of the medical care information, and a graph (graph structure) including the plurality of entities as nodes is generated as the structured medical care information.
Example 3: A plurality of entities is extracted from at least a part of the medical care information, and a table (tabular format) including the plurality of entities as data items is generated as the structured medical care information.

### First Generation Unit 13

The first generation unit 13 generates input information including the structured medical care information and a query. Here, the input information is input to a generative model to be described later. The input information may be expressed as a prompt or the like. The structured medical care information is generated by the structuring unit 12 as described above. Meanwhile, the query may be determined in advance, or may be based on information acquired by the acquisition unit 11.

For example, the acquisition unit 11 may acquire criterion information on a clinical trial, and the first generation unit 13 may generate the input information including the criterion information as the query. Alternatively, the acquisition unit 11 may acquire criterion information on a clinical trial, the structuring unit 12 may generate structured criterion information by structuring at least a part of the criterion information, and the first generation unit 13 may generate the input information including the structured criterion information as the query.

The first generation unit 13 may generate the input information that includes text extracted from the medical record (electronic medical record) or the medical care information as it is (that is, without structuring using the structuring unit 12). For example, the first generation unit 13 may generate the input information that includes a sentence such as “there is a finding of xxx, so that examination yyy is required at the next examination” extracted from the medical care information as it is without structuring.

### Second Generation Unit 14

The second generation unit 14 generates an answer to the query related to the subject with reference to output from the generative model having received the input information. Here, the generative model may be a machine-learned language model such as a large language models (LLMs), a generative model using a graph database, or another model, for example. Here, examples of the generative model using a graph database include graph retrieval augmented generation (GraphRAG), but the present exemplary example embodiment is not limited to the GraphRAG. For example, the above generative model may be provided in the information processing apparatus 1, or may be provided in another apparatus communicably connected to the information processing apparatus 1.

The second generation unit 14 may directly use output from the generative model having received the input information as the answer, or may generate the answer by processing the output from the generative model having received the input information.

For example, the second generation unit 14 may refer to output from the generative model having received the input information to generate the answer that includes information on how much the subject is adapted to the clinical trial.

### Effect of Information Processing Apparatus 1

As described above, the information processing apparatus 1 uses a configuration of:
acquiring medical care information on one or more subjects;
generating structured medical care information by structuring at least a part of the medical care information;
generating input information including the structured medical care information and a query; and
generating an answer to the query related to the one or more subjects with reference to output from a generative model having received the input information. As described above, the information processing apparatus 1 generates the structured medical care information from the medical care information and generates the answer with reference to the output from the generative model having received the input information including the structured medical care information, and thus can perform estimation related to the subject (patient) with high accuracy.

### Flow of Information Processing Method S1

Next, a flow of an information processing method S1 according to the present exemplary example embodiment will be described with reference to FIG. 2. FIG. 2 is a flowchart illustrating the flow of the information processing method S1. As illustrated in FIG. 2, the information processing method S1 includes step (processing) S11 of acquiring medical care information, step (processing) S12 of generating structured medical care information from the medical care information, step (processing) S13 of generating input information including the structured medical care information and a query, and step (processing) S14 of generating an answer to the query.

### Step S11

In step S11, the acquisition unit 11 acquires medical care information on one or more subjects.

The acquisition unit 11 has been more specifically described above, and thus is not described here.

### Step S12

In step S12, the structuring unit 12 generates structured medical care information by structuring at least a part of the medical care information acquired by the acquisition unit 11 in step S11. The structuring unit 12 has been more specifically described above, and thus is not described here.

### Step S13

In step S13, the first generation unit 13 generates input information including the structured medical care information and the query. The first generation unit 13 has been more specifically described above, and thus is not described here.

### Step S14

In step S14, the second generation unit 14 generates an answer to the query related to the subject with reference to output from the generative model having received the input information. The second generation unit 14 has been more specifically described above, and thus is not described here.

### Effect of Information Processing Method S1

As described above, the information processing method S1 uses a configuration of:
acquiring medical care information on one or more subjects; generating structured medical care information by structuring at least a part of the medical care information; generating input information including the structured medical care information and a query; and generating an answer to the query related to the one or more subjects with reference to output from a generative model having received the input information. The configuration described above achieves an effect similar to that of the information processing apparatus 1.

### Second Example Embodiment

A second exemplary example embodiment that is an example of an example embodiment of the present invention will be described in detail with reference to the drawings. Components that have the same functions as the components described in the above-described exemplary example embodiment are denoted by the same reference signs, and will not be described as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be adopted in the other exemplary example embodiments included in the present disclosure within a range in which no particular technical problem occurs.

### Configuration of Information Processing System 100A

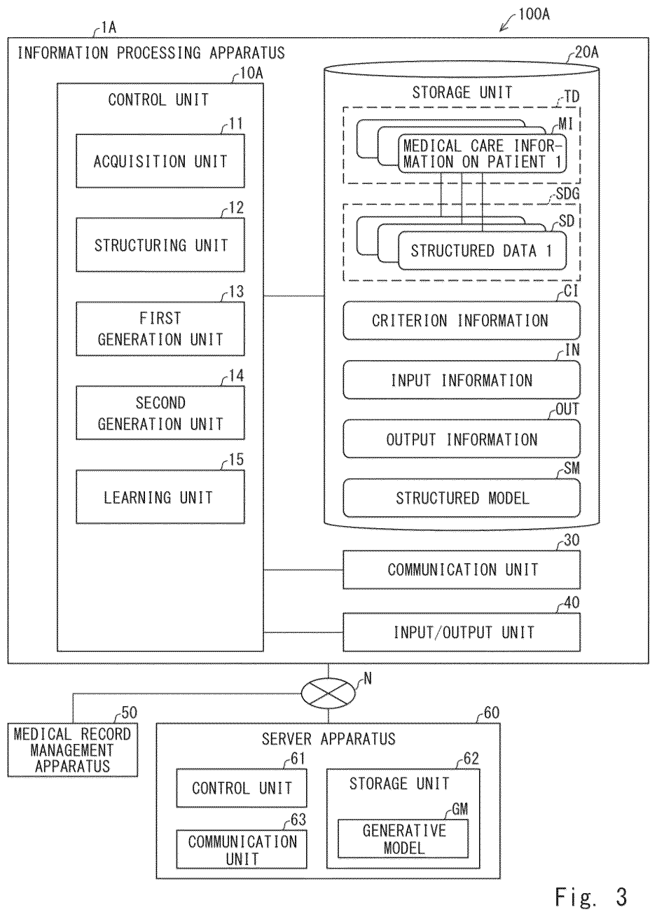

A configuration of an information processing system 100A according to the present exemplary example embodiment will be described with reference to FIG. 3. FIG. 3 is a block diagram illustrating a configuration of the information processing system 100A. As illustrated in FIG. 3, the information processing system 100A includes an information processing apparatus 1A, a medical record management apparatus 50 and a server apparatus 60 that are connected to the information processing apparatus 1A through a network N. Here, the network N has a specific configuration that is not limited to that of the present exemplary example embodiment, and available examples of the network N include a wireless local area network (LAN), a wired LAN, a wide area network (WAN), a public line network, a mobile data communication network, and a combination of these networks. The configuration illustrated in FIG. 3 is merely an example. For example, the information processing apparatus 1A may include some or all of the configurations of the medical record management apparatus 50 and the server apparatus 60. More specifically, the information processing apparatus 1A may include a storage unit 20A to be described later, in which at least one of an electronic medical record and a generative model GM to be described later is stored.

### Medical Record Management Apparatus 50

The medical record management apparatus 50 manages electronic medical records of a plurality of subjects (patient, clinical trial candidate). The electronic medical record of each subject includes medical care information on the subject. The electronic medical record of each patient or the medical care information on the patient is acquired and referred to by the information processing apparatus 1A.

### Server Apparatus 60

As illustrated in FIG. 3, the server apparatus 60 includes a control unit 61, a storage unit 62, and a communication unit 63. The communication unit 63 communicates with an apparatus outside the server apparatus 60. For example, the communication unit 63 communicates with the information processing apparatus 1A provided in the information processing system 100A. The communication unit 63 transmits data supplied from the control unit 61 to the information processing apparatus 1A, and supplies data received from the information processing apparatus 1A to the control unit 61. The data received by the communication unit 63 from the information processing apparatus 1A can include input information (prompt) generated by the information processing apparatus 1A. The data provided by the communication unit 53 to the information processing apparatus 1A can include output information generated by the generative model GM to be described later based on the input information.

The storage unit 62 stores the generative model GM. For example, the storage unit 62 stores a plurality of parameters defining the generative model GM. These parameters are learned in advance through machine learning (parameters subjected to update processing through machine learning), for example, and do not limit the present exemplary example embodiment. As the generative model GM, a large language model subjected to machine learning may be used. Alternatively, a generative model using a graph database or another model may be used as the generative model GM. Here, examples of the generative model using a graph database include graph retrieval augmented generation (GraphRAG), but the present exemplary example embodiment is not limited to the GraphRAG.

The control unit 61 acquires information generated by the generative model GM by using the generative model GM. For example, the control unit 61 acquires output information (generation result) generated by the generative model GM based on the input information (prompt) received from the information processing apparatus 1A, the prompt including structured medical care information to be described later. The output information is provided to the information processing apparatus 1A by using the communication unit 63.

Although the server apparatus 60 is exemplified as an apparatus separate from the information processing apparatus 1A in the present exemplary example embodiment, this example does not limit the present exemplary example embodiment. The information processing apparatus 1A may include a control unit having a function as the control unit 61 provided in the server apparatus 60 or a generative model execution unit in the control unit 61. Similarly, the information processing apparatus 1A may include a storage unit that stores the generative model GM stored in the storage unit 62 provided in the server apparatus 60, thereby enabling the generative model GM to be executed by the information processing apparatus 1A itself.

### Configuration of Information Processing Apparatus 1A

Next, a configuration of the information processing apparatus 1A according to the present exemplary example embodiment will be described with reference to FIG. 3. As illustrated in FIG. 3, the information processing apparatus 1A includes a control unit 10, a storage unit 20, a communication unit 30, and an input/output unit 40.

### Communication Unit 30

The communication unit 30 communicates with an apparatus outside the information processing apparatus 1A. For example, the communication unit 30 communicates with the medical record management apparatus 50 and the server apparatus 60. The communication unit 30 transmits data supplied from the control unit 10 to the server apparatus 60, and supplies data received from the medical record management apparatus 50 and the server apparatus 60 to the control unit 10. The data received by the communication unit 30 from the medical record management apparatus 50 may include electronic medical records or medical care information on each of a plurality of subjects (patient, clinical trial candidate). The data transmitted from the communication unit 30 to the server apparatus 60 can include input information (prompt) generated by the first generation unit 13 described later. The data received by the communication unit 30 from the server apparatus 60 may include a generation result (output information) generated by the generative model GM based on the input information.

### Input/Output Unit 40

The input/output unit 40 includes at least any one of input/output apparatuses such as a keyboard, a mouse, a display, a printer, and a touch panel. Alternatively, the input/output unit 40 may be connected to an input/output device such as a keyboard, a mouse, a display, a printer, or a touch panel. This configuration allows the input/output unit 40 to receive inputs of various types of information to the information processing apparatus 1A from a connected input device. The input/output unit 40 also outputs various types of information to a connected output device under control of the control unit 10. Examples of the input/output unit 40 include an interface such as a universal serial bus (USB).

### Storage Unit 20

The storage unit 20 stores various types of data to be referred to by the control unit 10 and various types of data generated by the control unit 10. For example, the storage unit 20 stores the following:
Medical care information MI of each subject (patient, clinical trial candidate);
Structured data SD;
Criterion information CI;
Input information IN;
Output information OUT; and
Structured model SM.

Here, the medical care information MI may include various types of information extracted from an electronic medical record of each subject. For example, the medical care information MI may include information on the subject, such as an initial medical interview, a progress record, an image interpretation report, and a nursing record. However, these examples do not limit the present exemplary example embodiment. Then, a medical care information group including the medical care information MI on a plurality of subjects may be expressed as target data TD.

The structured data SD is generated from the medical care information MI on each subject by the structuring unit 12 described later. A specific example of the structured data SD will be described later. Then, a data group including the structured data SD related to the plurality of subjects may be expressed as a structured data group SDG.

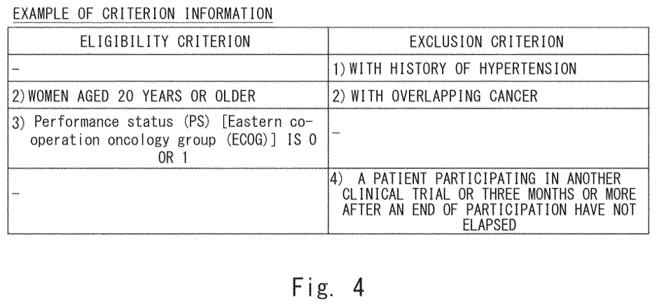

For example, the criterion information CI relates to clinical trial. The criterion information CI may include at least any one of an eligibility criterion (criterion to be satisfied as a clinical trial subject) and an exclusion criterion (criterion not to be satisfied as a clinical trial subject), for example. FIG. 4 illustrates an example of the criterion information CI.

The example illustrated in FIG. 4 shows an exclusion criterion 1) “with history of hypertension” set as the criterion information CI on clinical trial 1). As the criterion information CI on clinical trial 2), an eligibility criterion 2) “women aged 20 years or older” and an exclusion criterion 2) “with overlapping cancer” are set. Then, as the criterion information CI on clinical trial 3), an eligibility criterion 3) “performance status (PS) [Eastern cooperation oncology group (ECOG)] is 0 or 1” is set. Additionally, as the criterion information CI on clinical trial 4), an exclusion criterion 4) “a patient participating in another clinical trial or three months or more after an end of participation have not elapsed” is set. However, these examples do not limit the present exemplary example embodiment.

The input information IN is generated by the first generation unit 13 to be described later, and is used as an input to the generative model GM described above. A specific example of the input information IN will be described later.

The output information OUT is generated by the second generation unit 14 to be described later with reference to output from the generative model GM having received the input information IN. A specific example of the output information OUT will be described later.

The structured model SM is referred to by the first generation unit 13, and is used to generate the structured data SD described above. A plurality of structured models SM may be provided.

A specific example of the structured model SM will be described later.

### Control Unit 10

As illustrated in FIG. 3, the control unit 10 includes the acquisition unit 11, the structuring unit 12, the first generation unit 13, the second generation unit 14, and a learning unit 15.

### Acquisition Unit 11

The acquisition unit 11 acquires the medical care information MI on one or more subjects. Here, the medical care information may include various types of information extracted from the electronic medical record of each subject managed by the medical record management apparatus 50. The specific example of the medical care information MI has been described above, so that the description will not be duplicated.

### Structuring Unit 12

The structuring unit 12 generates structured medical care information by structuring at least a part of the medical care information MI acquired by the acquisition unit 11. Here, the structured medical care information is an example of the structured data SD described above. As in the first exemplary example embodiment, the structuring unit 12 can be configured to generate information in a data format of at least any one of a triplet, a graph, and a table as the structured medical care information.

The structuring unit 12 may be configured to select one or more structured models from the plurality of structured models SM learned (machine learned) using learning data different from each other with reference to the criterion information on the clinical trial, and generate the structured medical care information using the selected one or more structured models SM. A more specific processing example performed by the structuring unit 12 will be described later with reference to the drawings.

### First Generation Unit 13

The first generation unit 13 generates the input information IN including the structured medical care information and a query.

Here, the input information IN is to be input to the generative model GM as described above. The query may be determined in advance, or may be based on information acquired by the acquisition unit 11.

For example, the acquisition unit 11 may acquire the criterion information CI regarding a clinical trial, and the first generation unit 13 may generate the input information IN including the criterion information CI as the query. Alternatively, the acquisition unit 11 may acquire the criterion information CI regarding the clinical trial, the structuring unit 12 may generate structured criterion information by structuring at least a part of the criterion information CI, and the first generation unit 13 may generate the input information including the structured criterion information as the query. As with the structured medical care information described above, the structured criterion information can be generated here as information in a data format of at least one of a triplet, a graph, and a table.

The first generation unit 13 may generate the input information IN that includes text extracted from the electronic medical record or the medical care information MI as it is (that is, without structuring using the structuring unit 12). For example, the first generation unit 13 may generate the input information that includes a sentence such as “there is a finding of xxx, so that examination yyy is required at the next examination” extracted from the medical care information as it is without structuring.

### Second Generation Unit 14

The second generation unit 14 generates an answer (output information OUT) to the query related to the subject with reference to the output from the generative model GM having received the input information IN. The second generation unit 14 may directly use output from the generative model GM having received the input information IN as the answer (output information OUT), or may generate the answer (output information OUT) by processing the output from the generative model GM having received the input information IN. For example, the second generation unit 14 may refer to output from the generative model GM having received the input information IN to generate the answer (output information OUT) that includes information on how much the subject is adapted to the clinical trial.

### Learning Unit 15

The learning unit 15 trains the structured model SM. For example, the learning unit 15 trains each of the plurality of structured models SM (machine learning) using learning data for each clinical department or each category. A specific example of learning processing performed by the learning unit 15 will be described later with reference to the drawings.

### Processing Flow Example 1 Performed by Information Processing System 100A

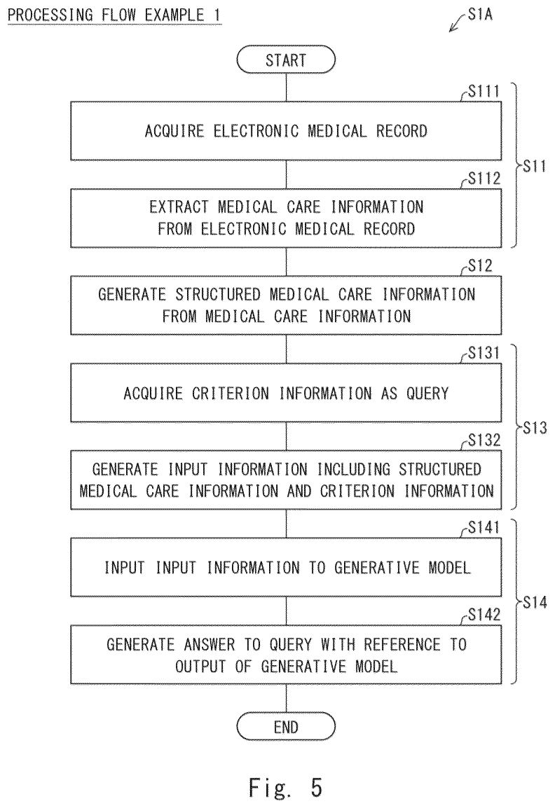

Next, a processing flow example 1 performed by the information processing system 100A will be described with reference to FIG. 5. FIG. 5 is a flowchart illustrating the processing flow example 1 (information processing method S1A) performed by the information processing system 100A.

### Steps S111 and S112

In step S111, the acquisition unit 11 acquires electronic medical records of one or more subjects from the medical record management apparatus 50. Then, the acquisition unit 111 extracts the medical care information MI on the subject from the electronic medical records in step S112. Steps S111 and S112 can correspond to step S11 described in the first exemplary example embodiment.

### Step S12

Subsequently, the structuring unit 12 generates structured medical care information (structured data SD) from the medical care information MI using the structured model SM in step S12.

### Steps S131 and S132

Subsequently, the acquisition unit 11 acquires the criterion information CI related to the clinical trial as a query in step S131. Then, the first generation unit 13 generates the input information IN including the structured medical care information (structured data SD) generated in step S12 and the query, in step S132. Steps S131 and S132 can correspond to step S13 described in the first exemplary example embodiment.

### Steps S141 and S142

Subsequently, the second generation unit 14 inputs the input information IN generated in step S132 to the generative model GM through the communication unit 30 in step S141. Then, the second generation unit 14 acquires output (generation result) from the generative model GM having received the input information IN through the communication unit 30 in step S142. The second generation unit 14 then generates the output information OUT with reference to the output (generation result). Here, the output information OUT includes an answer to the query acquired in step S131.

For example, the output information OUT includes information on how much the subject is adapted to the clinical trial.

As described above, the information processing apparatus 1A uses a configuration of:
acquiring the medical care information MI on one or more subjects (steps S111 and S112);
generating the structured medical care information (structured data SD) by structuring at least a part of the medical care information MI (step S12);
generating the input information IN including the structured medical care information and a query (steps S131 and S132); and
generating an answer (output information OUT) to the query related to the one or more subjects with reference to output from the generative model GM having received the input information IN (steps S141 and S142). As described above, the information processing apparatus 1A generates the structured medical care information from the medical care information MI and generates the answer with reference to the output from the generative model GM having received the input information IN including the structured medical care information, and thus can perform estimation related to the subject (patient, clinical trial candidate) with high accuracy. Additionally, the input information IN including the structured medical care information is used, so that processing cost caused by the generative model GM can be reduced.

Although a configuration has been conventionally known in which medical care information on a patient is directly input to a large language model, such a configuration may cause all information input to the large language model not to be processed due to too large amount of the information. As a result, accuracy may decrease. In contrast, the information processing apparatus 1A configured as described above generates the structured medical care information from the medical care information MI and generates the answer with reference to the output from the generative model GM having received the input information IN including the structured medical care information, so that the processing is suitably performed in the generative model GM. Thus, estimation related to the subject (patient, clinical trial candidate) can be performed with high accuracy.

### Data Processing Example 1

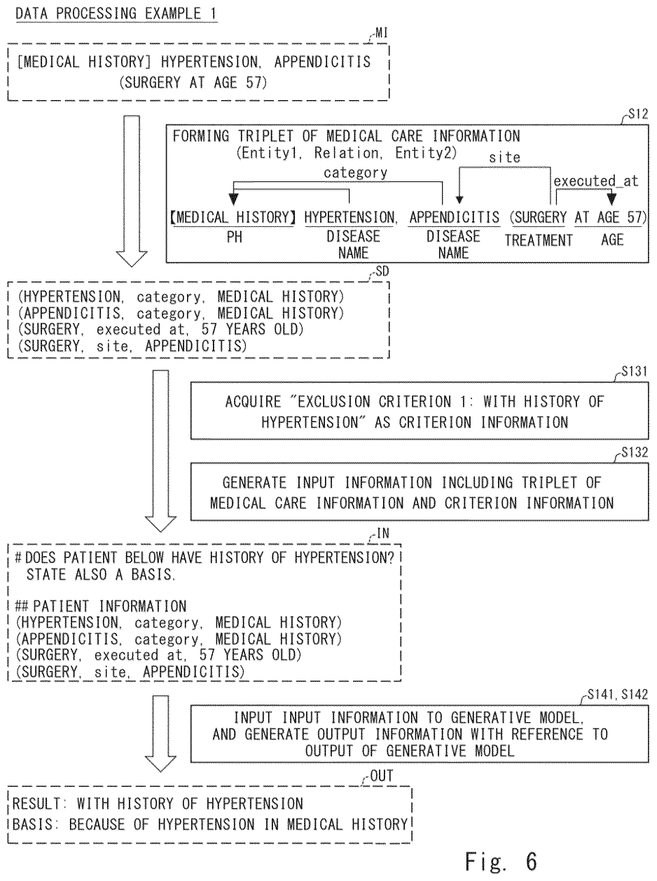

Subsequently, a more specific data processing example 1 performed by the information processing apparatus 1A will be described with reference to FIG. 6. The example illustrated in FIG. 6 first shows the medical care information MI acquired by the acquisition unit 11, which indicates “Medical history: Hypertension, appendicitis (surgery at age 57)” (corresponding to steps S111 and S112 described above).

Then, the structuring unit 12 extracts a plurality of entities (hypertension, appendicitis, 57 years old, surgery) from the medical care information MI, and generates a plurality of triplets including the plurality of entities and a relation between the plurality of entities as the structured medical care information SD (corresponding to step S12 described above), the plurality of triplets including:
(Hypertension, category, medical history);
(Appendicitis, category, medical history);
(Surgery, executed at, 57 years old); and
(Surgery, site, appendicitis).

As in the above example, the triplet generated by the structuring unit 12 is here configured as (Entity 1, Relation, Entity 2) with a first entity (Entity 1), a second entity (Entity 2), and a relation between the first entity and the second entity (Relation) as elements. Here, the relation may be an oriented concept or a concept including no orientation.

The structuring unit 12 may perform the structuring processing described above using the structured model SM described above or may perform the structuring processing described above using the generative model GM.

Subsequently, the first generation unit 13 acquires “exclusion criterion 1: with a history of hypertension” as the criterion information CI (corresponding to step S131 described above), and generates the input information IN including the structured medical care information SD and the query in accordance with the criterion information CI (corresponding to step S132 described above).

More specifically, the first generation unit 13 generates the input information IN including “Does a patient below have a history of hypertension?” as a query in accordance with the criterion information CI as illustrated in FIG. 6. The query here may include an instruction to include a basis in an answer (“state also a basis” in FIG. 6) as illustrated in FIG. 6. As illustrated in FIG. 6, the input information IN generated by the first generation unit 13 includes structured medical care information SD generated by the structuring unit 12 as patient information, the structured medical care information SD including:
(Hypertension, category, medical history);
(Appendicitis, category, medical history);
(Surgery, executed at, 57 years old); and
(Surgery, site, appendicitis). As described above, the first generation unit 13 also may perform processing of including text extracted from the electronic medical record or the medical care information MI as it is (that is, without structuring performed by the structuring unit 12).

Subsequently, the second generation unit 14 inputs the input information IN to the generative model GM as a prompt (corresponding to step S141 described above). Then, the second generation unit 14 acquires output (generation result and estimation result) of the generative model GM. The output information OUT is then generated with reference to the acquired generation result (corresponding to step S142 described above). The example illustrated in FIG. 6 shows that the second generation unit 14 obtains a generation result acquired by the generative model GM, the generation result including:
Result: with a history of hypertension; and
Basis: Because of hypertension in medical history,
the second generation unit 14 using the generation result as it is as the output information OUT. As described above, the present example shows the query that includes instruction information indicating that an answer needs to include also a basis, and the answer (output information OUT) generated by the second generation unit 14, the answer including information on how much the subject is adapted to the clinical trial and a basis of the information, as information for supporting decision-making of the user.

By generating a triplet including an entity extracted from the medical care information MI as the structured medical care information SD as in the present example, estimation accuracy using the generative model GM can be improved and processing cost caused by the generative model GM can be reduced. By instructing the generative model GM to include a basis of estimation in a generation result (estimation result) as in the present example, decision-making of the user (doctor or medical worker) can be supported by the output information OUT.

### Data Processing Example 2

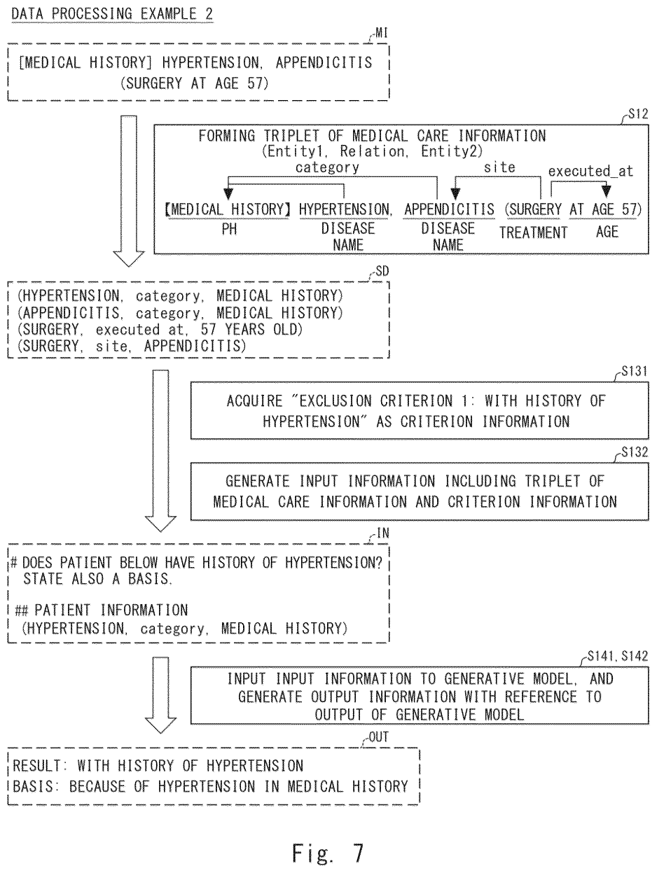

Subsequently, a more specific data processing example 2 performed by the information processing apparatus 1A will be described with reference to FIG. 7. The example illustrated in FIG. 7 is different from the data processing example 1 illustrated in FIG. 6 in points below, and is identical in other points to the data processing example 1.

That is, the data processing example 2 illustrated in FIG. 7 shows that processing is performed when the first generation unit 13 generates the input information IN in step S132, the processing including:
extracting only a triplet related to the criterion information CI from a plurality of triplets generated by the structuring unit 12; and
including the extracted triplet in the input information IN as patient information. More specifically, the first generation unit 13 refers to the criterion information CI and extracts a triplet (hypertension, category, medical history) related to the criterion information CI from a plurality of triplets generated by the structuring unit 12 to generate the input information IN including the extracted triplet as the patient information as illustrated in FIG. 7, the plurality of triplets including:
(Hypertension, category, medical history);
(Appendicitis, category, medical history);
(Surgery, executed at, 57 years old); and
(Surgery, site, appendicitis).

As described above, by extracting the structured medical care information related to the criterion information CI (in other words, the query) from the plurality of pieces of structured medical care information generated by the structuring unit 12 to generate the input information IN including the extracted structured medical care information as the patient information, estimation accuracy using the generative model GM can be improved and calculation cost caused by the generative model GM can be reduced.

### Data Processing Example 3

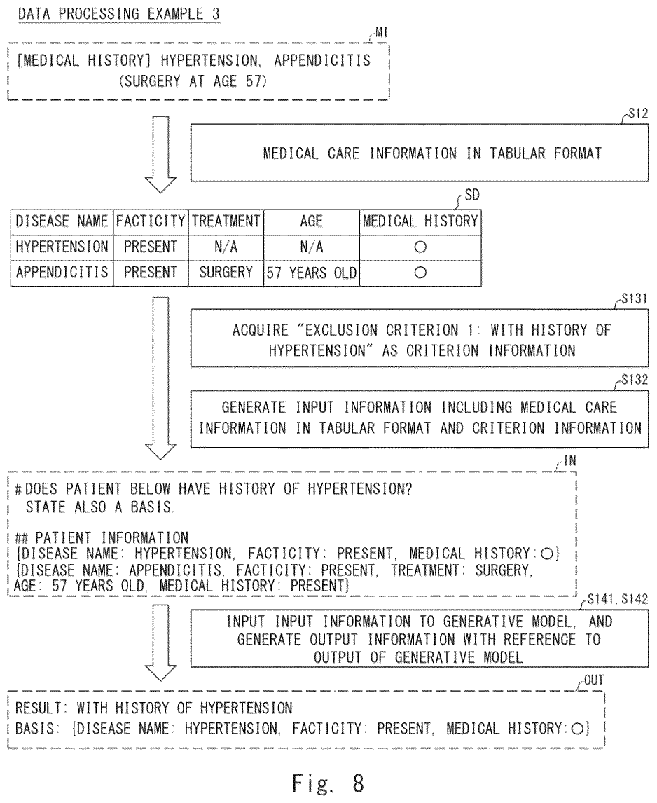

Subsequently, a more specific data processing example 3 performed by the information processing apparatus 1A will be described with reference to FIG. 8. The example illustrated in FIG. 8 is different from the data processing example 1 illustrated in FIG. 6 in points below, and is identical in other points to the data processing example 1.

That is, the data processing example 3 illustrated in FIG. 8 shows step S12 in which the structuring unit 12 extracts a plurality of entities from at least a part of the medical care information MI to generate a table (tabular format) including the plurality of entities as data items as the structured medical care information SD.

Then, the data processing example 3 shows the first generation unit 13 that generates the input information IN including the structured medical care information SD in the table format, and that inputs the input information IN to the generative model GM (corresponding to steps S131 and S141) as illustrated in FIG. 8. More specifically, the first generation unit 13 generates the input information IN as patient information, and inputs the input information IN to the generative model GM, the input information IN including:
{Disease name: Hypertension, Facticity: Present, Medical history}; and
{Disease name: Appendicitis, Facticity: Present, Treatment: Surgery, Age: 57 years old, Medical history}.

Even in a case where structured medical care information in a table format is generated as the structured data SD as in the present example, estimation accuracy using the generative model GM can be improved and calculation cost caused by the generative model GM can be reduced.

### Data Processing Example 4

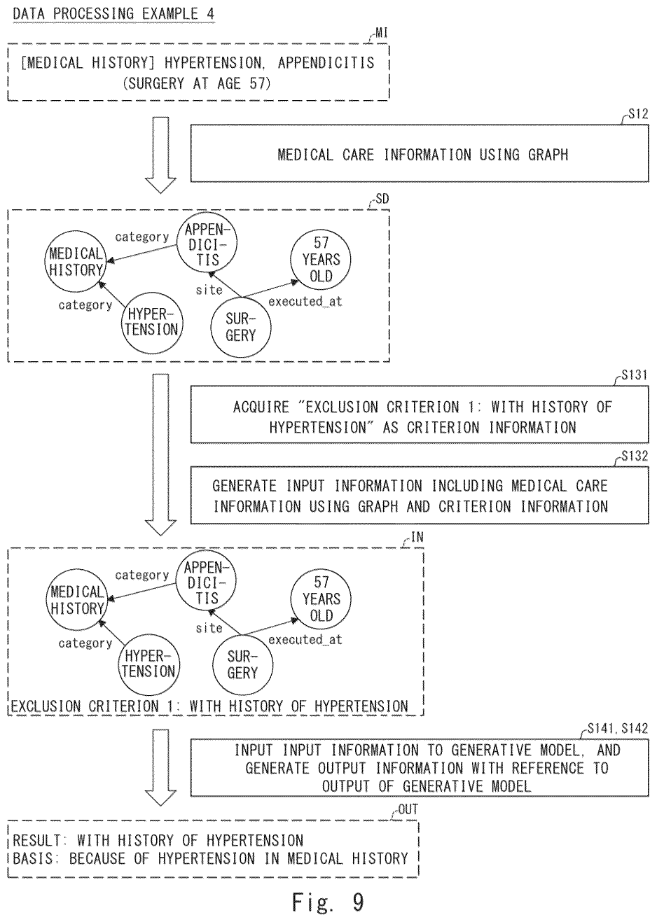

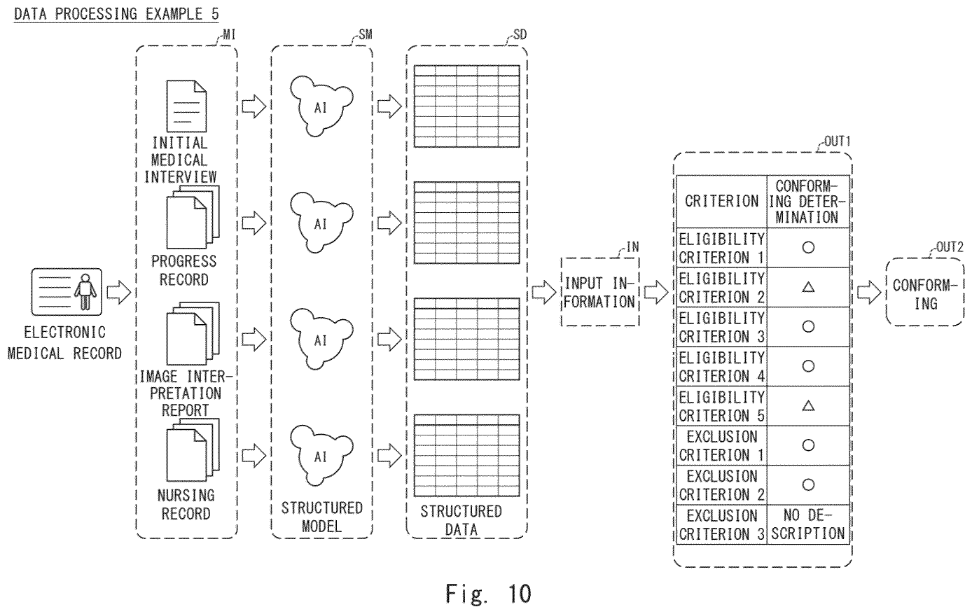

Subsequently, a more specific data processing example 4 performed by the information processing apparatus 1A will be described with reference to FIG. 9. The example illustrated in FIG. 9 is different from the data processing example 1 illustrated in FIG. 6 in points below, and is identical in other points to the data processing example 1.

That is, the data processing example 4 illustrated in FIG. 9 shows step S12 in which the structuring unit 12 extracts a plurality of entities from at least a part of the medical care information MI to generate a graph (graph structure) including the plurality of entities as nodes as the structured medical care information SD.

Then, the data processing example 4 shows the first generation unit 13 that generates the input information IN including the structured medical care information SD in the graph format, and that inputs the input information IN to the generative model GM (corresponding to steps S131 and S141) as illustrated in FIG. 9. The graph generated by the structuring unit 12 may be a directed graph in which each link has orientation, an undirected graph in which each link does not have orientation, or a combination thereof. In a case where the structuring unit 12 generates a directed graph, orientation of each link is determined in accordance with relevance between entities (between nodes) extracted from the medical care information MI.

Even in a case where structured medical care information in a graph format is generated as the structured data SD as in the present example, estimation accuracy using the generative model GM can be improved and calculation cost caused by the generative model GM can be reduced.

### Data Processing Example 5

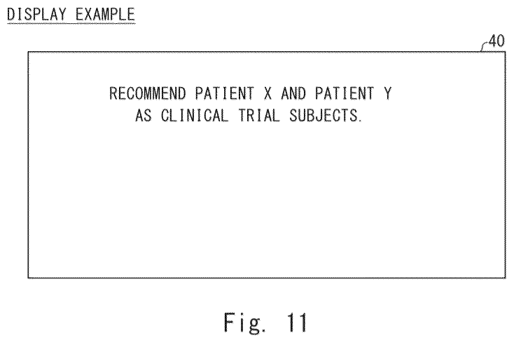

Subsequently, a more specific data processing example 5 performed by the information processing apparatus 1A will be described with reference to FIG. 10. The example illustrated in FIG. 10 shows that the acquisition unit 11 obtains an initial medical interview, a progress record, an image interpretation report, and a nursing record as the medical care information MI from an electronic medical record managed by the medical record management apparatus 50. Then, the structuring unit 12 individually applies the structured model SM to each of the initial medical interview, the progress record, the image interpretation report, and the nursing record to generate the structured data SD.

For example, the structuring unit 12 may be configured to select the structured model SM to be applied to the initial medical interview included in the medical care information MI from a plurality of models with reference to the medical care information MI. Here, the structured model SM applied to the initial medical interview is obtained by machine learning using learning data including sets of a plurality of initial medical interviews and a correct answer label (correct answer label related to structuring) attached to each initial medical interview, for example.

Similarly, the structuring unit 12 may be configured to select the structured model SM to be applied to the progress record included in the medical care information MI from a plurality of models with reference to the medical care information MI. Here, the structured model SM applied to the progress record is obtained by machine learning using learning data including sets of a plurality of progress records and a correct answer label (correct answer label related to structuring) attached to each progress record, for example. The same applies to the image interpretation report and the nursing record.

Learning of each structured model SM described above can be performed in advance by the learning unit 15. In other words, the learning unit 15 may be configured to cause each of the plurality of structured models SM to perform machine learning using learning data for each category (learning data different from each other). Here, examples of the category include each of categories such as the initial medical interview, the progress record, the image interpretation report, and the nursing record described above.

The learning unit 15 also may be configured to cause each of the plurality of structured models SM to perform machine learning using learning data for each clinical department. This configuration enables preparing individual learning data (learning data different from each other) for each of clinical departments such as cardiovascular internal medicine, gastrointestinal medicine, and respiratory internal medicine, for example, thereby causing the structured model SM for each clinical department to be learned using the learning data.

As described above, the structuring unit 12 can be configured to select one or more structured models from the plurality of structured models SM machine-learned using learning data different from each other with reference to the criterion information CI regarding the clinical trial, and generate the structured medical care information using the selected one or more structured models SM.

The present example also shows the input information IN generated by the first generation unit 13, the input information IN including the structured medical care information SD (e.g., in a table format) generated from each of the initial medical interview, the progress record, the image interpretation report, and the nursing record. Here, each table may be in an independent table format or an integrated table format. One table and another table each may include information contradictory to each other. In preparation for the tables, the first generation unit 13 may be configured to give priority to each table (describe the priority of each table in the input information IN) so that the generative model GM refers to each table in accordance with the priority. Alternatively, the input information IN may include instruction information indicating that consistency is also verified in the generative model GM, such as “In a case where one table and another table each include information contradictory to each other, an answer needs to be generated after determination of which information is appropriate”.

As illustrated in FIG. 10, the present example shows the second generation unit 14 that generates output information OUT1 including an estimation result (determination result) for each of a plurality of criteria included in the criterion information CI for a certain subject using the generative model GM. The second generation unit 14 in the present example also generates a final answer (output information) OUT2 indicating whether the subject is adapted to the clinical trial with reference to the output information OUT1.

More specifically, the second generation unit 14 may be configured as illustrated in FIG. 10 to perform processing of:
referring to a determination result for each criterion (eligibility criteria 1 to 5, exclusion criteria 1 to 3) included in the output information OUT1, the determination result being acquired by the generative model GM;
determining whether there is a non-conforming criterion; and
generating output information OUT2 indicating that the subject is adapted to the clinical trial in a case where there is no non-conforming criterion. The processing of the present example enables further improvement in estimation accuracy using the generative model GM and further reduction in calculation cost caused by the generative model GM.

### Display Example

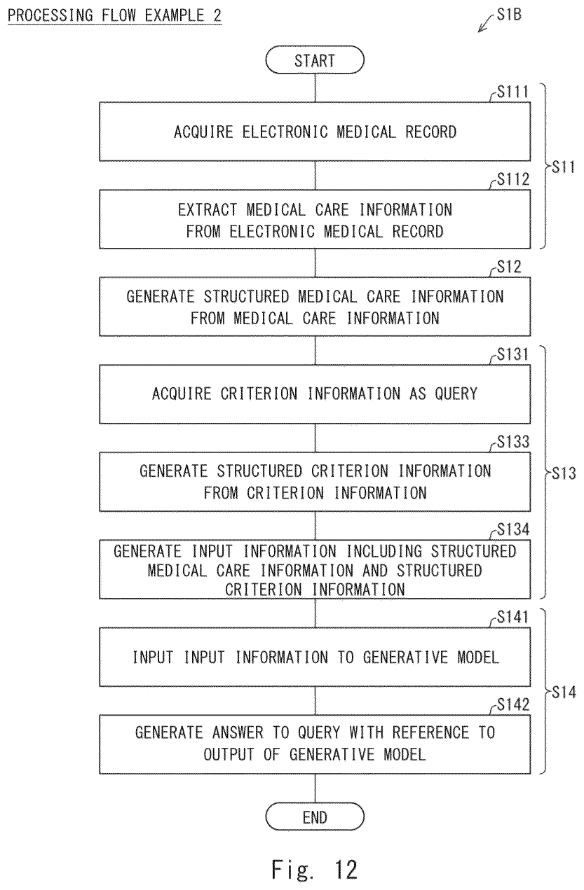

FIG. 11 is a display example in a case where the output information OUT generated by the second generation unit 14 is displayed using a display provided in the input/output unit 40. As illustrated in FIG. 11, the second generation unit 14 may display the output information OUT using the input/output unit 40, the output information OUT including information regarding which clinical trial candidate among a plurality of clinical trial candidates is adapted to the clinical trial. The output information OUT according to the example is also an example of information for supporting the decision-making of the user (doctor or medical worker).

The second generation unit 14 may be configured to generate the output information OUT including not only a generation result generated by the generative model GM but also supplementary information. For example, the output information OUT may include information on a subject determined to be adapted to the clinical trial in the generation result generated by the generative model GM, the information including intention (desire or not desire) of the subject to participate in the clinical trial and being acquired from the electronic medical record or another database. However, the supplementary information is not limited to this example.

### Processing Flow Example 2 Performed by Information Processing System 100A)

Subsequently, a processing flow example 2 performed by the information processing system 100A will be described with reference to FIG. 12. FIG. 12 is a flowchart illustrating the processing flow example 2 (information processing method S1B) processing performed by the information processing system 100A.

As illustrated in FIG. 12, the processing according to the present example is different from Example 1 of the flow of the processing illustrated in FIG. 5 in a point below, and is similar to Example 1 of the flow of the processing in other points. That is, the processing according to the present example includes steps S133 and S134 instead of step S132 illustrated in FIG. 5.

### Step S133

In step S133, structured criterion information is generated from the criterion information CI acquired in step S131. For example, the structured criterion information may be generated by the structuring unit 12 or may be generated using the generative model GM. As with the structured medical care information SD described above, the structured criterion information can be generated in a data format of at least any one of a triplet, a graph, and a table, for example. More specifically, processing below may be performed.
Example 1: A plurality of entities is extracted from at least a part of the criterion information CI, and one or more triplets including the plurality of entities and a relation between the plurality of entities are generated as the structured criterion information.
Example 2: A plurality of entities is extracted from at least a part of the criterion information CI, and a graph (graph structure) including the plurality of entities as nodes is generated as the structured criterion information.
Example 3: A plurality of entities is extracted from at least a part of the criterion information CI, and a table (tabular format) including the plurality of entities as data items is generated as the structured criterion information.

### Step S134

Subsequently, the first generation unit 13 generates the input information IN in step S134, the input information IN including the structured medical care information (structured data SD) generated in step S12 and the structured criterion information generated in step S133 as a query.

As described above, the structured criterion information is generated from the criterion information CI and the input information IN including the structured criterion information as a query is generated in the processing according to the present example, thereby improving estimation accuracy using the generative model GM and reducing calculation cost caused by the generative model GM.

### Third Example Embodiment

A third exemplary example embodiment that is an example of an example embodiment of the present invention will be described in detail with reference to the drawings. Components that have the same functions as the components described in the above-described exemplary example embodiment are denoted by the same reference signs, and will not be described as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be adopted in the other exemplary example embodiments included in the present disclosure within a range in which no particular technical problem occurs.

### Configuration of Information Processing System 100B

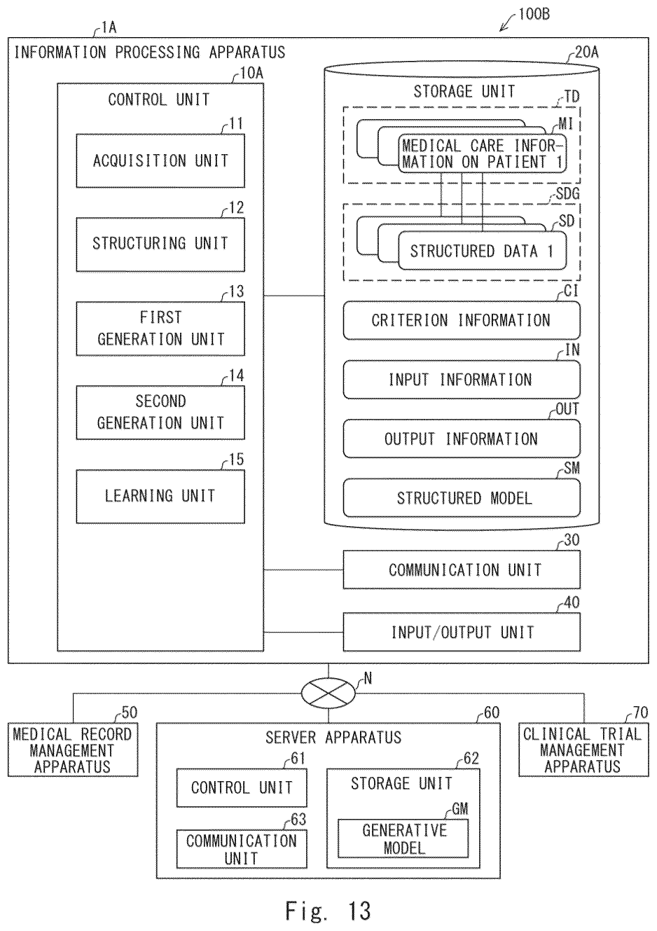

A configuration of an information processing system 100B according to the present exemplary example embodiment will be described with reference to FIG. 13. FIG. 13 is a block diagram illustrating the configuration of the information processing system 100B. As illustrated in FIG. 13, the information processing system 100B includes an information processing apparatus 1A, and a medical record management apparatus 50, a server apparatus 60, and a clinical trial management apparatus 70 connected to the information processing apparatus 1A through a network N. The information processing apparatus 1A, the medical record management apparatus 50, and the server apparatus 60 are similar to those of the second exemplary example embodiment and have been already described, so that the description will not be duplicated.

### Clinical Trial Management Apparatus 70

The clinical trial management apparatus 70 manages implementation of a clinical trial. For example, the clinical trial management apparatus 70 acquires or manages the following:
Data on clinical trial type;
Data on drugs used in each clinical trial;
Data on candidates for each clinical trial (clinical trial candidates);
Pre-clinical trial data on subjects of each clinical trial (clinical trial subjects);
In-clinical trial data on subjects of each clinical trial; and
Post-clinical trial data on subjects of each clinical trial. The clinical trial management apparatus 70 may be configured to generate output data (clinical trial report or the like) with reference to the above-described data, for example. The information processing system 100B may include a plurality of information processing apparatuses 1A. Such a system configuration can be used for collectively managing the information processing apparatus 1A installed for each hospital, for example. The clinical trial management apparatus 70 in such a configuration may be configured to perform processing of:
receiving supply of output information from the information processing apparatus 1A of each hospital; and
aggregating output information from each hospital, and outputting a candidate list, for example.

In the present exemplary example embodiment, the second generation unit 14 outputs the generated output information OUT to the clinical trial management apparatus 70. Here, the second generation unit 14 is preferably configured to generate the output information OUT including information (patient ID or the like) identifying a clinical trial candidate adapted to a target clinical trial.

Then, the clinical trial management apparatus 70 performs processing related to the clinical trial with reference to the output information OUT supplied from the information processing apparatus 1A. The clinical trial management apparatus 70 acquires data on the clinical trial candidate from the medical record management apparatus 50 with reference to an ID of the clinical trial candidate included in the output information OUT, for example.

The information processing system 100B according to the present exemplary example embodiment uses a configuration of:
acquiring medical care information on one or more subjects;
generating structured medical care information by structuring at least a part of the medical care information;
generating input information including the structured medical care information and a query;
generating an answer to the query about the one or more subjects (information on whether the one or more subjects are adapted to a clinical trial) with reference to output from a generative model having received the input information; and
supplying output information OUT including the answer to the clinical trial management apparatus 70. Thus, the clinical trial can be suitably performed on subjects adapted to the clinical trial.

### Implementation Example by Software

Some or all of the functions of the information processing apparatuses 1, 1A, and 1B (referred to below also as “each of the above apparatuses”) may be implemented by hardware such as an integrated circuit (IC chip) or may be implemented by software.

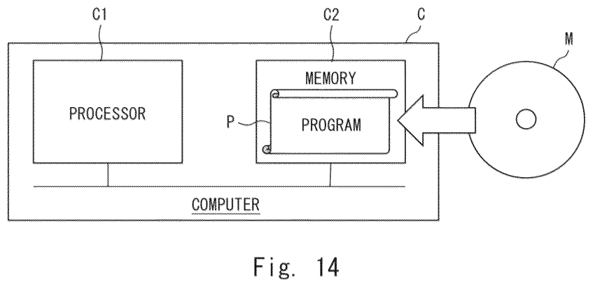

For the latter, each of the above apparatuses is implemented by a computer that executes a command of a program that is software for implementing each function, for example. FIG. 14 illustrates an example of such a computer (referred to below as a computer C). FIG. 14 is a block diagram illustrating a hardware configuration of the computer C functioning as each of the above apparatuses.

The computer C includes at least one processor C1 and at least one memory C2. The memory C2 records a program P for causing the computer C to operate as each of the above apparatuses. The processor C1 in the computer C reads out the program P from the memory C2 and executes the program P to implement each function of each of the above apparatuses.

Available examples of the processor C1 include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Available examples of the memory C2 include a flash memory, a hard disk drive (HDD), a solid state drive (SSD), and a combination thereof.

The computer C may further include a random access memory (RAM) for expanding the program P at the time of execution and temporarily storing various types of data. The computer C may further include a communication interface for transmitting and receiving data to and from another apparatus. The computer C may further include an input/output interface for connecting input/output devices such as a keyboard, a mouse, a display, and a printer.

The program P can be recorded in a tangible recording medium M that is non-transitory and readable by the computer C. Available examples of the recording medium M include a tape, a disk, a card, a semiconductor memory, and a programmable logic circuit.

The computer C can acquire the program P using the recording medium M described above. The program P can be transmitted using a transmission medium. Available examples of the transmission medium include a communication network and a broadcast wave. The computer C can also acquire the program P using the transmission medium described above.

Each of the above functions of each of the above apparatuses may be implemented by a single processor provided in a single computer, may be achieved in cooperation with a plurality of processors provided in a single computer, or may be achieved in cooperation with a plurality of processors provided in respective computers. The program for causing each of the above apparatuses to implement corresponding one of the above functions may be stored in a single memory provided in a single computer, may be stored in a distributed manner in a plurality of memories provided in a single computer, or may be stored in a distributed manner in a plurality of memories provided in respective computers.

### Supplementary Note Matter A

The present disclosure includes a technique described in each of Supplementary Notes below. However, the present invention is not limited to the technique described in each of Supplementary Notes below, and various modifications can be made within the scope described in the claims.

### Supplementary Note A1

An information processing apparatus including:
acquisition means for acquiring medical care information on one or more subjects;
structuring means for generating structured medical care information by structuring at least a part of the medical care information;
first generation means for generating input information including the structured medical care information and a query; and
second generation means for generating an answer to the query related to the one or more subjects with reference to an output from a generative model having received the input information.

### Supplementary Note A2

The information processing apparatus described in Supplementary Note A1, wherein
the acquisition means further acquires criterion information on a clinical trial,
the first generation means generates the input information including the criterion information as the query, and
the second generation means generates the answer including information on how much the subject is adapted to the clinical trial.

### Supplementary Note A3

The information processing apparatus described in Supplementary Note A1, wherein
the acquisition means further acquires criterion information on a clinical trial,
the structuring means further generates structured criterion information by structuring at least a part of the criterion information,
the first generation means generates the input information including the structured criterion information as the query, and
the second generation means generates the answer including information on how much the subject is adapted to the clinical trial.

### Supplementary Note A4

The information processing apparatus described in Supplementary Note A2 or A3, wherein
the query includes instruction information indicating that an answer needs to include also a basis, and
the answer generated by the second generation means includes information on how much the subject is adapted to the clinical trial and a basis of the information, as information for supporting decision-making of a user.

### Supplementary Note A5

The information processing apparatus described in any one of Supplementary Notes A2 to A4, wherein
the structuring means extracts a plurality of entities from at least a part of the medical care information, and
the structuring means generates one or more triplets including the plurality of entities and a relation between the plurality of entities, or a graph including the plurality of entities as nodes, as the structured medical care information.

### Supplementary Note A6

The information processing apparatus described in any one of Supplementary Notes A2 to A4, wherein
the structuring means extracts a plurality of entities from at least a part of the medical care information, and
the structuring means generates a table including the plurality of entities as data items as the structured medical care information.

### Supplementary Note A7

The information processing apparatus described in any one of Supplementary Notes A2 to A6, wherein
the structuring means selects one or more structured models from a plurality of structured models that is machine learned using learning data different from each other with reference to the criterion information on the clinical trial, and
the structuring means generates the structured medical care information using the selected one or more structured models.

### Supplementary Note A8

The information processing apparatus described in Supplementary Note A7, further including learning means for causing each of the plurality of structured models to perform machine learning using learning data for each clinical department or each category.

### Supplementary Note Matter B

The present disclosure includes a technique described in each of Supplementary Notes below. However, the present invention is not limited to the technique described in each of Supplementary Notes below, and various modifications can be made within the scope described in the claims.

### Supplementary Note B1

An information processing method including:
acquisition processing of acquiring medical care information on one or more subjects by using at least one processor;
structuring processing of generating structured medical care information by structuring at least a part of the medical care information by using the at least one processor;
first generation processing of generating input information including the structured medical care information and a query by using the at least one processor; and
second generation processing of generating an answer to the query related to the one or more subjects with reference to an output from a generative model having received the input information by using the at least one processor.

### Supplementary Note B2

The information processing method described in Supplementary Note B1, wherein
the acquisition processing is performed to further acquire criterion information on a clinical trial by using the at least one processor,
the first generation processing is performed to generate the input information including the criterion information as the query, and
the second generation processing is performed to generate the answer including information on how much the subject is adapted to the clinical trial.

### Supplementary Note B3

The information processing method described in Supplementary Note B1, wherein
the acquisition processing is performed to further acquire criterion information on a clinical trial by using the at least one processor,
the structuring processing is performed to further generate structured criterion information by structuring at least a part of the criterion information by using the at least one processor,
the first generation processing is performed to generate the input information including the structured criterion information as the query, and
the second generation processing is performed to generate the answer including information on how much the subject is adapted to the clinical trial.

### Supplementary Note B4

The information processing method described in Supplementary Note B2 or B3, wherein
the query includes instruction information indicating that an answer needs to include also a basis, and
the answer generated in the second generation processing includes information on how much the subject is adapted to the clinical trial and a basis of the information, as information for supporting decision-making of a user.

### Supplementary Note B5

The information processing method described in any one of Supplementary Notes B2 to B4, wherein
the at least one processor extracts a plurality of entities from at least a part of the medical care information in the structuring processing, and
the at least one processor generates one or more triplets including the plurality of entities and a relation between the plurality of entities, or a graph including the plurality of entities as nodes, as the structured medical care information.

### Supplementary Note B6

The information processing method described in any one of Supplementary Notes B2 to B4, wherein
the structuring processing is performed to extract a plurality of entities from at least a part of the medical care information by using the at least one processor, and
the structuring processing is performed to generate a table including the plurality of entities as data items as the structured medical care information by using the at least one processor.

### Supplementary Note B7

The information processing method described in any one of Supplementary Notes B2 to B6, wherein
the structuring processing is performed to select one or more structured models from a plurality of structured models that is machine learned using learning data different from each other with reference to the criterion information on the clinical trial by using the at least one processor, and
the structuring processing is performed to generate the structured medical care information using the selected one or more structured models by using the at least one processor.

### Supplementary Note B8

The information processing method described in Supplementary Note B7, further including learning processing of causing each of the plurality of structured models to perform machine learning using learning data for each clinical department or each category by using the at least one processor.

### Supplementary Note Matter C

The present disclosure includes a technique described in each of Supplementary Notes below. However, the present invention is not limited to the technique described in each of Supplementary Notes below, and various modifications can be made within the scope described in the claims.

### Supplementary Note C1

An information processing program for causing a computer to function as an information processing apparatus, the program causing the computer to function as:
acquisition means for acquiring medical care information on one or more subjects;
structuring means for generating structured medical care information by structuring at least a part of the medical care information;
first generation means for generating input information including the structured medical care information and a query; and
second generation means for generating an answer to the query related to the one or more subjects with reference to an output from a generative model having received the input information.

### Supplementary Note C2

The information processing program described in Supplementary Note C1, wherein
the acquisition means further acquires criterion information on a clinical trial,
the first generation means generates the input information including the criterion information as the query, and
the second generation means generates the answer including information on how much the subject is adapted to the clinical trial.

### Supplementary Note C3

The information processing program described in Supplementary Note C1, wherein
the acquisition means further acquires criterion information on a clinical trial,
the structuring means further generates structured criterion information by structuring at least a part of the criterion information,
the first generation means generates the input information including the structured criterion information as the query, and
the second generation means generates the answer including information on how much the subject is adapted to the clinical trial.

### Supplementary Note C4

The information processing program described in Supplementary Note C2 or C3, wherein
the query includes instruction information indicating that an answer needs to include also a basis, and
the answer generated by the second generation means includes information on how much the subject is adapted to the clinical trial and a basis of the information, as information for supporting decision-making of a user.

### Supplementary Note C5

The information processing program described in any one of Supplementary Notes C2 to C4, wherein
the structuring means extracts a plurality of entities from at least a part of the medical care information, and
the structuring means generates one or more triplets including the plurality of entities and a relation between the plurality of entities, or a graph including the plurality of entities as nodes, as the structured medical care information.

### Supplementary Note C6

The information processing program described in any one of Supplementary Notes C2 to C4, wherein
the structuring means extracts a plurality of entities from at least a part of the medical care information, and
the structuring means generates a table including the plurality of entities as data items as the structured medical care information.

### Supplementary Note C7

The information processing program described in any one of Supplementary Notes C2 to C6, wherein
the structuring means selects one or more structured models from a plurality of structured models that is machine learned using learning data different from each other with reference to the criterion information on the clinical trial, and
the structuring means generates the structured medical care information using the selected one or more structured models.

### Supplementary Note C8

The information processing program described in Supplementary Note C7, further causing the computer to function as learning means for causing each of the plurality of structured models to perform machine learning using learning data for each clinical department or each category.

### Supplementary Note Matter D

The present disclosure includes a technique described in each of Supplementary Notes below. However, the present invention is not limited to the technique described in each of Supplementary Notes below, and various modifications can be made within the scope described in the claims.

### Supplementary Note D1

An information processing apparatus including at least one processor,
the at least one processor performing:
acquisition processing of acquiring medical care information on one or more subjects;
structuring processing of generating structured medical care information by structuring at least a part of the medical care information;
first generation processing of generating input information including the structured medical care information and a query; and
second generation processing of generating an answer to the query related to the one or more subjects with reference to an output from a generative model having received the input information.

The information processing apparatus may further include a memory. The memory may store a program for causing the at least one processor to perform each processing described above.

### Supplementary Note D2

The information processing apparatus described in Supplementary Note D1, wherein
the at least one processor further acquires criterion information on a clinical trial in the acquisition processing,
the input information including the criterion information is generated as the query in the first generation processing, and
the answer including information on how much the subject is adapted to the clinical trial is generated in the second generation processing.

### Supplementary Note D3

The information processing apparatus described in Supplementary Note D1, wherein
the at least one processor further acquires criterion information on a clinical trial in the acquisition processing,
the at least one processor further generates structured criterion information by structuring at least a part of the criterion information in the structuring processing,
the input information including the structured criterion information is generated as the query in the first generation processing, and
the answer including information on how much the subject is adapted to the clinical trial is generated in the second generation processing.

### Supplementary Note D4

The information processing apparatus described in Supplementary Note D2 or D3, wherein
the query includes instruction information indicating that an answer needs to include also a basis, and
the answer generated in the second generation processing includes information on how much the subject is adapted to the clinical trial and a basis of the information, as information for supporting decision-making of a user.

### Supplementary Note D5

The information processing apparatus described in any one of Supplementary Notes D2 to D4, wherein
the at least one processor extracts a plurality of entities from at least a part of the medical care information in the structuring processing, and
the at least one processor generates one or more triplets including the plurality of entities and a relation between the plurality of entities, or a graph including the plurality of entities as nodes, as the structured medical care information.

### Supplementary Note D6

The information processing apparatus described in any one of Supplementary Notes D2 to D4, wherein
the at least one processor extracts a plurality of entities from at least a part of the medical care information in the structuring processing, and
the at least one processor generates a table including the plurality of entities as data items as the structured medical care information in the structuring processing.

### Supplementary Note D7

The information processing apparatus described in any one of Supplementary Notes D2 to D6, wherein
the at least one processor selects one or more structured models from a plurality of structured models that is machine learned using learning data different from each other with reference to the criterion information on the clinical trial in the structuring processing, and
the at least one processor generates the structured medical care information using the selected one or more structured models in the structuring processing.

### Supplementary Note D8

The information processing apparatus described in Supplementary Note D7, wherein the at least one processor further performs learning processing of causing each of the plurality of structured models to perform machine learning using learning data for each clinical department or each category.

### Supplementary Note Matter E

The present disclosure includes a technique described in each of Supplementary Notes below. However, the present invention is not limited to the technique described in each of Supplementary Notes below, and various modifications can be made within the scope described in the claims.

### Supplementary Note E1

A non-transitory storage medium storing an information processing program for causing a computer to function as an information processing apparatus, the program causing the computer to perform processing including:
acquisition processing of acquiring medical care information on one or more subjects;
structuring processing of generating structured medical care information by structuring at least a part of the medical care information;
first generation processing of generating input information including the structured medical care information and a query; and
second generation processing of generating an answer to the query related to the one or more subjects with reference to an output from a generative model having received the input information.

1. An information processing apparatus comprising:
at least one memory storing instructions; and
at least one processor configured to execute the instructions to;
acquire medical care information on one or more subjects;
generate structured medical care information by structuring at least a part of the medical care information;
generate input information including the structured medical care information and a query; and
generate an answer to the query related to the one or more subjects with reference to an output from a generative model having received the input information.

2. The information processing apparatus according to claim 1, wherein the at least one processor is further configured to execute the instructions to:
acquire criterion information on a clinical trial,
generate the input information including the criterion information as the query, and
generate the answer including information on how much the subject is adapted to the clinical trial.

3. The information processing apparatus according to claim 1, wherein the at least one processor is further configured to execute the instructions to:
acquire criterion information on a clinical trial,
generate structured criterion information by structuring at least a part of the criterion information,
generate the input information including the structured criterion information as the query, and
generate the answer including information on how much the subject is adapted to the clinical trial.

4. The information processing apparatus according to claim 2, wherein
the query includes instruction information indicating that an answer needs to include also a basis, and
the answer includes information on how much the subject is adapted to the clinical trial and a basis of the information, as information for supporting decision-making of a user.

5. The information processing apparatus according to claim 1, wherein the at least one processor is further configured to execute the instructions to:
extract a plurality of entities from at least a part of the medical care information, and
generate one or more triplets including the plurality of entities and a relation between the plurality of entities, or a graph including the plurality of entities as nodes, as the structured medical care information.

6. The information processing apparatus according to claim 1, wherein the at least one processor is further configured to execute the instructions to:
extract a plurality of entities from at least a part of the medical care information, and
generate t a table including the plurality of entities as data items as the structured medical care information.

7. The information processing apparatus according to claim 1, wherein the at least one processor is further configured to execute the instructions to:
select one or more structured models from a plurality of structured models that is machine learned using learning data different from each other with reference to the criterion information on the clinical trial, and
generate the structured medical care information using the selected one or more structured models.

8. The information processing apparatus according to claim 7, wherein the at least one processor is further configured to execute the instructions to:
cause each of the plurality of structured models to perform machine learning using learning data for each clinical department or each category.

9. An information processing method that uses one or more processors to perform processing comprising:
acquiring medical care information on one or more subjects;
generating structured medical care information by structuring at least a part of the medical care information;
generating input information including the structured medical care information and a query; and
generating an answer to the query regarding the one or more subjects with reference to output from a generative model having received the input information.

10. The information processing method according to claim 9, wherein
the acquisition processing is performed to further acquire criterion information on a clinical trial by using the at least one processor,
the first generation processing is performed to generate the input information including the criterion information as the query, and
the second generation processing is performed to generate the answer including information on how much the subject is adapted to the clinical trial.

11. The information processing method according to claim 9, wherein
the acquisition processing is performed to further acquire criterion information on a clinical trial by using the at least one processor,
the structuring processing is performed to further generate structured criterion information by structuring at least a part of the criterion information by using the at least one processor,
the first generation processing is performed to generate the input information including the structured criterion information as the query, and
the second generation processing is performed to generate the answer including information on how much the subject is adapted to the clinical trial.

12. The information processing method according to claim 10, wherein
the query includes instruction information indicating that an answer needs to include also a basis, and
the answer generated in the second generation processing includes information on how much the subject is adapted to the clinical trial and a basis of the information, as information for supporting decision-making of a user.

13. The information processing method according to claim 9, wherein
the at least one processor extracts a plurality of entities from at least a part of the medical care information in the structuring processing, and
the at least one processor generates one or more triplets including the plurality of entities and a relation between the plurality of entities, or a graph including the plurality of entities as nodes, as the structured medical care information.

14. The information processing method to claim 9, wherein
the structuring processing is performed to extract a plurality of entities from at least a part of the medical care information by using the at least one processor, and
the structuring processing is performed to generate a table including the plurality of entities as data items as the structured medical care information by using the at least one processor.

15. The information processing method to claim 9, wherein
the structuring processing is performed to select one or more structured models from a plurality of structured models that is machine learned using learning data different from each other with reference to the criterion information on the clinical trial by using the at least one processor, and
the structuring processing is performed to generate the structured medical care information using the selected one or more structured models by using the at least one processor.

16. The information processing method to claim 15, further including learning processing of causing each of the plurality of structured models to perform machine learning using learning data for each clinical department or each category by using the at least one processor.

17. A non-transitory storage medium storing an information processing program for causing a computer to function as an information processing apparatus, the program causing the computer to perform processing including:
acquisition processing of acquiring medical care information on one or more subjects;
structuring processing of generating structured medical care information by structuring at least a part of the medical care information;
first generation processing of generating input information including the structured medical care information and a query; and
second generation processing of generating an answer to the query related to the one or more subjects with reference to an output from a generative model having received the input information.
